(19) United States
(12) Patent Application Publication
(10) Pub. No.: US 2005/0113646 A1
(43) Pub. Date: May 26, 2005

(54) Method and apparatus for evaluation of sleep disorders

(76) Inventors: John G. Sotos, Palo Alto, CA (US); John L. Branscum JR., Belmont, CA (US)

Correspondence Address: Kenneth M. Kaslow;c/o Apneos Corporation, #41, 2033 Ralston Ave., Belmont, CA, 94002, US

(21) Appl. No.: 10/721,115

(22) Filed: Nov. 24, 2003

(51) Int. Cl. A61B 5/00

(52) U.S. Cl. 600/300

(57) ABSTRACT

A method and apparatus for the evaluation of sleep disorders is disclosed. Two sensors are used to diagnose and usefully characterize abnormal sleep breathing, a sensor of tracheal vibration, and a sensor of axial body position. The two sensors are attached to the patient in locations substantially adjacent to one another. In a preferred embodiment, these two sensors can be physically combined into a single unit, thereby increasing further the simplicity and reliability of attaching the sensors to the patient. The unit is applied to a patient near a tracheal segment, preferably at a suprasternal notch location. The position sensor has two axes of sensitivity that are at angles to each other so that the sensor may determine which of four positions it is in relative to gravity. When attached to the patient, the sensor is oriented so that it may be determined whether the patient is oriented in a supine, prone, left lateral decubitus, or right lateral decubitus position. Data are recorded from both sensors concurrently, preferably over a period of time of several hours, and stored in a recording device, preferably containing a non-volatile memory, so that the data may be reviewed later for diagnosis and characterization.

### BACKGROUND OF THE INVENTION

1. Field of the Invention

The invention relates generally to a method and apparatus for obtaining physiological data from a patient during sleep. More specifically, the invention relates to a method and apparatus for the diagnosis and characterization of sleep disorders by recording at least the tracheal sounds of the patient and the patient's orientation with respect to gravity.

2. Related Art

The present invention relates to a simple and low-cost method and apparatus for assessing respiratory ventilation. It may find use in diagnosing and characterizing sleep disorders, primarily, but not limited to, sleep disordered breathing.

Sleep disordered breathing (SDB) has been defined simply as “abnormal breathing patterns that disrupt sleep” (Bond. Oral Maxillofacial Surg Clin N Am. 2002; 14:293-296). More complicated and controversial definitions of SDB appear in the medical literature (See, e.g., respectively, Quan, Littner, AASM. Sleep. 1999; 22:662, 665-666, 667-689). A variety of breathing patterns (or “events”) may disrupt sleep, including, but not limited to, apneas, hypopneas, and respiratory effort-related arousals. SDB has been sub-typed into different syndromes, including, but not limited to, obstructive sleep apnea (OSA), central sleep apnea, and upper airway resistance syndrome. The definitions of these syndromes typically refer to threshold values for the occurrence of SDB events during sleep. For example, OSA is commonly defined in adults as the summed occurrence of 5 or more obstructive apneas or obstructive hypopneas per hour of sleep. The more general syndrome, “sleep apnea,” may be defined similarly, but without the requirement for obstructive types of apneas and hypopneas. A lower threshold, i.e. a threshold occurrence-rate of obstructive events that is lower than 5 per hour, is sometimes used to define OSA in children. Event rates less than 5 per hour may also be linked with adverse health states in adults (Peppard. N Engl J. Med. 2000; 342:1378-1384). Snoring may or may not accompany various types of breathing events and SDB syndromes, or it may occur in isolation. Herein, we consider snoring as a type of SDB.

There is evidence that SDB is a common occurrence. Young et al (Am J Respir Crit Care Med. 2002; 165:1219-1239) estimate “that roughly 1 of every 5 adults has at least mild OSA and 1 of every 15 has at least moderate OSA,” implying that over 30 million people in the United States suffer from sleep apnea. It is generally accepted that tens of millions of people in the United States snore at least occasionally.

SDB, and OSA in particular, may have adverse health consequences. Diagnosing and treating these conditions is, therefore, often desirable. Many types of SDB are treatable. Several types of treatment are available for OSA, including, but not limited to: continuous positive airway pressure (CPAP) and other types of positive airway pressure, surgery (e.g. uvulopalatopharyngoplasty (UPPP)), oral appliances (e.g. mandibular advancement devices), weight loss, positional therapy, and nasal decongestants.

There is, however, also evidence that at least certain types of SDB are under-diagnosed. For example, Young et al (Sleep. 1997; 20: 705-6) estimated that about 82% of men and 93% of women with symptomatic sleep apnea are not diagnosed as such.

According to Li and Flemons (Clin Chest Med. 2003; 24:283-295) the polysomnogram (PSG) is the “widely accepted reference standard for the diagnosis of sleep apnea.” PSGs normally record data from a plurality of sensors attached to a sleeping patient, often on the order of 15 sensors.

PSGs may be performed in an “attended” or “unattended” fashion. Writing for the American Sleep Disorders Association in 1997, Chesson et al. state that “[a]n attended study requires the constant presence of a trained individual who can monitor for technical adequacy, patient compliance, and relevant patient behavior” (Chesson. Sleep. 1997; 20:406-422).

Attended PSGs are usually expensive (e.g. on the order of $1,500 to $3,000) and, because attended PSGs are normally performed in a medical facility called a sleep laboratory, they are often inconvenient for the patient who sleeps away from home in the sleep laboratory. Li and Flemons (id.) describe the PSG as a “labor-intensive test [that] is time consuming and requires considerable technical expertise to perform and interpret . . . As a result, most health care jurisdictions have unacceptably long waiting times for sleep studies.” The negative aspects of the PSG may thus limit the ability of the sleep medicine community to diagnose the many persons who have SDB.

From data presented by Tachibana et al (Sleep. 2002; 25(Suppl.):A47), we estimate that perhaps as few as 1.2 million people in the United States devote the time and expense necessary to undergo polysomnography in a given year, out of the tens of millions that might benefit from diagnosis of SDB. Thus, there have been multiple attempts to lower the cost and increase the convenience of PSGs, while preserving diagnostic utility. As a simple example, a PSG unattended by a technician may be performed in a patient's home after a technician has attached the requisite sensors to the patient. However, a major concern with unattended PSGs is the loss of data that may occur if one or more sensors become dislodged or uncomfortable when there is no technician available to adjust or re-attach the sensor(s). For example, Goodwin et al report high levels of data loss in children undergoing unattended PSG and a high level of discomfort with some types of sensors (Goodwin. Sleep. 2001; 24:937-944).

To increase the reliability of data collection, sensor attachments have been improved (for example U.S. Pat. No. 6,201,982). Even so, a large number of sensors, alone, may cause considerable complexity and expense just to attach them properly. Thus, there have also been several attempts to introduce unattended diagnostic devices for sleep disorders that, in comparison with PSG, operate with a reduced number of sensors and/or sensor attachments and with a high reliability. These devices are often intended for use outside of a sleep laboratory.

A tradeoff with such reduced-sensor devices is that as the number of sensors is reduced, the amount of data collected may be reduced, possibly compromising diagnostic utility. In some sense, using a reduced-sensor device is akin to performing an unattended PSG wherein complete data loss occurs for one or more sensors, i.e. the PSG sensors that are not included in the reduced-sensor device. It is also noteworthy that some reduced-sensor devices employ one or more sensors that are not normally part of PSGs, such as a static-charge-sensitive bed.

Thus, in developing a reduced-sensor device for diagnosing sleep disorders such as SDB, the choice of sensor(s) and the method(s) of sensor attachment are critical. For example, in discussing home sleep testing Douglas remarks: “The choice of sensors to be used is open to considerable debate” (Douglas. Sleep Med Rev. 2003;7:53-59). In 1994 Ferber et al (Sleep. 1994; 17:378-392) noted that at least 27 devices had been manufactured for the diagnosis of OSA outside of a steep laboratory. Accordingly, reduced-sensor devices as a class employ a wide variety of sensors and sensor attachments, as illustrated by Ferber et al (id.), Ross et al (Sleep. 2000; 23:1-14), Flemons et al (Chest. 2003;124:1541-79 plus supplemental notes) and several prior patents and publications.

For example, U.S. Pat. No. 4,982,738 teaches a method and apparatus in which heart potentials (EKG) are measured by the use of three electrodes while a microphone applied near the larynx records breathing and snoring sounds.

Similarly, U.S. Pat. No. 5,275,159 teaches a method and apparatus in which a total of six attachments to the patient are required, three electrodes to measure the EKG, and three more attachments for a microphone, position pickup, and oximeter. In fact, while the described invention is claimed to avoid the need for a stay in a sleep laboratory, the patent mentions that at least some of the attachments “can be installed easily and in the correct position by medical technical assistants,” thus indicating that the device may not be suitable for home use.

U.S. patent application Ser. No. 040937 teaches a system attached to a patient's head for monitoring, at a minimum, pulse rate and oxygen saturation. In some embodiments a position sensor and microphone are added, again on the patient's head.

U.S. Pat. Nos. 5,671,733, 5,782,240, 5,879,313, 5,961,447 and 6,045,514 (same inventors) teach the use of respiratory sound data, arterial oxygen saturation data, and body position data in the diagnosis of snoring and sleep apnea.

A device called the Remmers Sleep Recorder (Remmers. Internet document, 2003), believed to be commercially available, utilizes three attachments to the patient's body to record snoring sounds, nasal airflow, oximetry, and whether the patient is supine or not.

Netzer et al (Chest. 2001; 120:625-633) review the utility of oximetry in diagnosing sleep disordered breathing.

Development of such devices continues, but as Li and Flemons (supra.) note, “[u]se of portable monitors at home for managing sleep apnea patients remains controversial and is not currently considered accepted practice by any specialty group.” Comments of Ross et al, published in 2000 (supra.), illustrate the unfilled need that still exists in diagnosing OSA: “A major problem in the field [of sleep apnea] is diagnosis: who to test, how to test, and what are the implications of test results regarding the risk of serious clinical sequelae? . . . The development of simpler and less costly alternatives for diagnosis or pre-PSG screening is highly desirable.”

Making a diagnosis, however, is normally not the ultimate goal in the medical management of patients with SDB. In many patients, diagnosis is only a step leading to treatment, preferably effective treatment. Unfortunately, not all diagnosed patients are effectively treated. There is evidence that one of the mainstays for treatment of OSA, CPAP, is under-utilized by patients for whom it is prescribed. For example, Grote et al (Eur Resp J. 2000; 16:921-7) reported a study in which subjects who accepted CPAP therapy had an adjusted CPAP compliance of only 56%. Grote et al further report that only about half of the subjects they studied accepted CPAP. There is evidence that UPPP surgery is effective in only about half of patients who undergo it for OSA (Walker-Engstrom. Chest. 2002; 121:739-746). Some authorities (e.g., Ferguson. Clin Chest Med. 2003; 24:355-364) believe that mandibular repositioning appliances (a type of oral appliance) should not be used as first-line therapy for patients with severe symptoms of OSA.

Because some therapies will be effective in some patients and less effective in others, it is reasonable to conclude that an important part of managing an individual patient with a particular type of SDB is choosing a therapy (or therapies) that will be effective for that particular patient. There are many possible variables on which the choice of therapy for a particular patient can be based, including the patient's age, the severity of the patient's disease, the presence of other medical conditions in the patient, the patient's occupation, conditions under which sleep breathing is disturbed, and so forth. The severity of the patient's disease may be reflected, for example, in the degree of symptoms experienced by the patient, in the occurrence rate of abnormal breathing events, and so forth. Thus, while a diagnostic device producing a simple count or occurrence rate of abnormal breathing events may partially characterize the severity of a patient's SDB, it may not provide adequate information to make the best therapeutic choice for the patient. It is therefore reasonable to conclude that diagnostic devices producing additional information useful in therapeutic decision-making may represent an improvement over devices that do not provide such additional information.

Thus, there is a need for a device that can be used in the unattended diagnosis of SDB with high reliability, having adequate diagnostic utility and, preferably, the ability to provide information useful in the management of patients found to have SDB. Because reliability can be, in part, determined by the number of sensors and/or sensor attachments that may be dislodged from the patient, a device with a small number of sensor attachments that are easy to apply and likely to remain fixed to the patient has potential advantages. Other useful features include low cost, the ability for the patient to self-apply the sensors, and a device that may be used in the patient's own home. It is the object of the present invention to provide a device with some or all of these advantages.

### SUMMARY OF THE INVENTION

The present invention is based upon the discovery that data from just two sensors can provide enough data to diagnose and usefully characterize many cases of abnormal sleep breathing. The two sensors are (1) a sensor of tracheal vibration, and (2) a sensor of axial body position. The two sensors are attached to the patient in locations substantially adjacent to one another.

In a preferred embodiment, these two sensors can be physically combined into a single unit, thereby increasing further the simplicity and reliability of attaching the sensors to the patient. The unit is applied to a patient near a tracheal segment, preferably at a suprasternal notch location. The position sensor has two axes of sensitivity that are at angles to each other so that the sensor may determine which of four positions it is in relative to gravity. When attached to the patient, the sensor is oriented so that it may be determined whether the patient is oriented in a supine, prone, left lateral decubitus, or right lateral decubitus position.

Data are recorded from both sensors concurrently, preferably over a period of time of several hours, and stored in a recording device, preferably containing a non-volatile memory, so that the data may be reviewed later for diagnosis and characterization.

### DESCRIPTION OF THE DRAWINGS

FIG. 1 is a diagram of a first embodiment of the present invention.

FIG. 2 shows how the gravity sensing devices indicate the position of the patient in one embodiment of the present invention.

FIGS. 3A and 3B show the top and bottom respectively of an adhesive patch for attaching one embodiment of the present invention to a patient.

FIG. 4 is a diagram showing how the adhesive patch of FIG. 3 is used with the illustrated embodiment of the present invention.

FIG. 5 shows an alternative method of utilizing an adhesive patch to attach an embodiment of the present invention to a patient.

### DETAILED DESCRIPTION OF THE PREFERRED EMBODIMENT

The present invention utilizes the discovery that data from just two sensors can provide enough information to diagnose and usefully characterize SDB in many patients. The two sensors are (1) a sensor of tracheal vibration, and (2) a sensor of axial body position. The two sensors are attached to the patient in locations substantially adjacent to one another. Adding to the value of the information that these sensors provide is the discovery that data from these two sensors can enable specific therapeutic decisions for a large proportion of patients with OSA and similar types of SDB. Another feature of the present invention is the combination of these two types of sensors into a single sensor attachment, as reducing the number of sensor attachments can be expected to increase the reliability and simplicity of SDB assessment.

As above, many devices in the art utilize snoring sounds in diagnosing SDB. Snoring sounds, however, are not the only vibrations that emanate from the airway of a sleeping patient. A great deal more information is available in non-snoring tracheal vibration, e.g. tracheal sounds. For example, the absence of tracheal sound within a certain frequency band may be an indication of apnea. As a further example, sounds of normal quiet breathing may also be present in tracheal sound. The tracheal sound intensity associated with normal quiet breathing may be low. Thus, distinguishing apnea (when it is characterized by absence of tracheal sound) from normal quiet breathing (when it is characterized by very low intensity tracheal sound) could be difficult if a sensor collecting tracheal vibration information were configured to detect only snoring sounds. (Although the source of snoring sounds is frequently in the soft tissues of the upper airway, some would classify them as tracheal vibration as well because they are readily detectable in recordings of tracheal sound.)

Snoring is often a loud noise, and although snores can be detected in tracheal vibrations, they generally can also be easily detected at quite a distance from their source. The placement of some snoring sensors is thus not tightly constrained. As noted earlier, non-snoring tracheal sounds may have low intensity. Thus, when deciding where to position a tracheal vibration sensor, having it near the trachea is often advantageous because it can result in pickup of a stronger signal. We call such positions near the trachea “peri-tracheal” positions. By our definition, a tracheal vibration sensor is able to usefully collect vibrations from the trachea or tracheal air space associated with normal quiet breathing, whereas a snoring sensor is not. A snoring sensor is able to usefully collect snoring vibrations, and may or may not be positioned peri-tracheally.

We consider tracheal vibration sensors usually to be superior to snoring sensors for detecting SDB, especially since some patients with SDB do not snore. Several reduced-sensor devices for diagnosing SDB have used tracheal vibration sensors, sometimes as the only sensor and sometimes as one of a plurality of sensors. We include microphones as a type of vibration sensor.

The present invention therefore distinguishes snoring sensors from tracheal vibration sensors, as summarized in the tables below.

| | Tracheal Vibration | Snoring |
|---|---|---|
| Includes snoring sound | Yes | Yes |
| Includes sound of normal quiet breathing | Yes | No |
| | Tracheal Vibration | Snoring |
| | Sensor | Sensor |
| Usefully detects snoring sound | Yes | Yes |
| Usefully detects sound of normal quiet | Yes | No |
| breathing | | |

Although tracheal vibration information can be used to diagnose some types of SDB, it alone often does not provide a sufficient characterization of the patient's disorder to allow appropriate management of the patient's disorder. The present invention improves upon this by combining the tracheal vibration sensor with a sensor for body position (i.e., the orientation of an axial portion of the patient's body with respect to an acceleration vector, normally the earth's gravity).

The present invention includes the recognition that positional effects in SDB may be particularly important because specific treatment decisions can be made when a person is found to have SDB with a significant positional component. For example, a patient who has SDB only when sleeping on his or her back may be treated with means to prevent sleeping in such a position. As an additional example, Cartwright (Sleep Med Rev. 2001;5:25-32) notes that a significant positional effect in SDB may be a predictor of successful treatment with an oral appliance. Furthermore, positional effects in SDB are likely to be common, if for no other reason than many snorers snore less when on their side than when on their back.

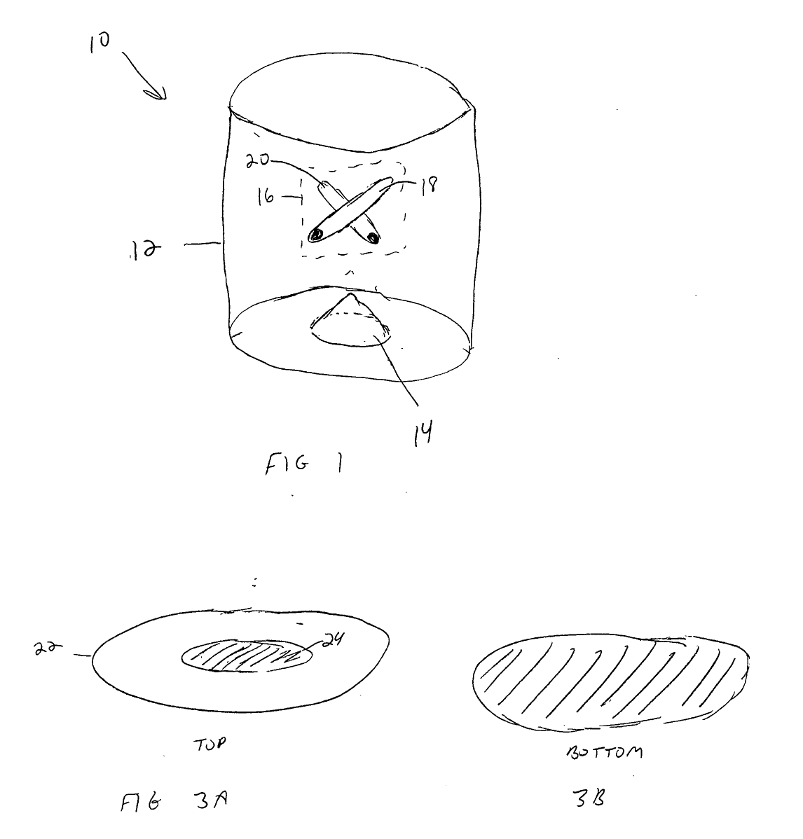

Referring now to FIG. 1, a first embodiment of the present invention is shown. A sensor 10 contains within a housing 12 a vibration sensor 14 and a position sensor 16. Position sensor 16 is comprised of two gravity-sensing devices 18 and 20 each having an axis of sensitivity with respect to gravity. The axes of sensitivity of the two gravity sensing devices 18 and 20 are at right angles to each other (when superpositioned on each other, as the actual devices are of course three-dimensional), and a plane containing the two superpositioned axes is at a right angle to the base of the housing 12.

One of the keys to the present invention is attaching both vibration sensor 14 and position sensor 16 to the patient in locations such that the sensors 14 and 16 are substantially adjacent to one another, and collecting information concurrently from both sensors 14 and 16 so that the tracheal vibration may be correlated with the patient's position.

In a preferred embodiment, vibration sensor 14 should capture as much of the patient's tracheal vibration as is useful and not be limited to snoring sounds. In a preferred embodiment, this is accomplished by using as vibration sensor 14 a microphone that has its sensitive portion near the bottom of the housing 12. A frequency sensitivity range of approximately 400 to 1000 hertz will ordinarily make the microphone capable of capturing a variety of breathing sounds, including not only snoring sounds, but also sounds of quiet breathing, quackling sounds and sighing sounds. (“Quackling” is an old word referring to sounds a person makes when being choked. In the current context, the word refers to sounds made as air passes through what is presumed to be a near-completely obstructed airway. The sounds are normally short and, to a human ear, reminiscent of choking sounds. They are sometimes called “struggle sounds.”) In some cases it may be desirable to use a microphone whose lower frequency limit of sensitivity is in the 20 to 50 hertz range, which will often enable detection of heart sounds, and will be useful for certain applications. Certain methods of snoring detection also rely on vibration sensors being sensitive to low frequencies.

In order for the vibration sensor 14 to record the greatest portion of the useful tracheal vibrations, it should be attached to the patient in a location where it can capture these tracheal vibrations, and not just snoring sounds. Thus, in a preferred embodiment the housing 12 is attached to the patient's body near a tracheal segment. Better results are often obtained if the housing 12 is attached to the patient's body in a pre-tracheal location, such as a suprasternal notch location or just below the cricoid cartilage.

With respect to position sensor 16, the goal is to determine the position of the patient's body so that it may be correlated to the tracheal vibrations or to physiology reflected in the tracheal vibrations. In a preferred embodiment, this is accomplished by using one or more gravity sensitive devices and by coupling the gravity sensitive device(s) to the patient's body. A gravity sensitive device is one that has an axis of sensitivity with respect to gravity, so that the device is capable of determining which end of the axis of sensitivity is closer to the center of the earth (the mathematical source of the gravity vector).

By coupling a gravity sensitive device to the patient's body in such a way that the device moves in concert with at least a portion of the patient's body, it is possible to correlate the information provided by the device with the position (with respect to gravity) of the body portion to which it is coupled. We use the term “body orientation information” to describe information indicative of the position, with respect to gravity, of the portion(s) of the body coupled to a gravity sensitive device. The coupling may be as simple as attaching a gravity sensitive device, or a housing containing such a device, to the skin. Such attachments may be removable; for example, the device or housing may be attached with adhesive such as that used in an adhesive bandage.

For questions related to airway patency during sleep, body orientation information for an axial portion of the body is often useful, since the airway and surrounding structures are generally axial structures themselves. Other axial structures include, but are not restricted to the head, the neck, and the chest. In a preferred embodiment, the gravity sensing devices 18 and 20 are tilt switches containing liquid mercury which connects contacts at one end or the other depending upon the orientation of the devices with respect to gravity, or accelerometers, as these seem to be an inexpensive, simple, and reliable means of implementation. It is also possible to use a single accelerometer chip that detects acceleration in one or more axes. However, as shown in the background art, there are other gravity sensing devices that will work, such as a polyhedron with an internal ball that makes contact in the various corners of the device as it changes position with respect to gravity. In some embodiments liquid mercury may be used instead of an internal ball, and vice versa. Other internal architectures of gravity-sensing devices are also possible, such as using a tethered mobile element.

In many cases, the most important data related to position is whether the patient is on his or her back or not, although the present invention may be used to determine other patient orientations as well. Whether or not the patient is on his or her back can actually be determined with a single gravity sensitive device coupled to an axial portion of the patient's body, configured to have an axis of sensitivity parallel to an antero-posterior axis of the patient's body. If, for example, the device is first coupled to the patient such that it shows one orientation while the patient is on his or her back, and subsequently indicates that its position with respect to gravity has shifted, i.e. that the end of the axis of sensitivity closest to the earth's center has changed, then there is an indication that the patient is no longer on his or her back.

However, it is often more informative if position sensor 16 can determine which of at least four positions it, and thus the patient, is in. In a preferred embodiment, these four positions are when the patient is on his or her back, front, left side or right side, also known as the supine, prone, left lateral decubitus, and right lateral decubitus positions, respectively. (An alternative that may satisfy some needs may be a sensor that can determine three positions, i.e. whether the patient is on his or her back, front, or either side.) This can be accomplished with two gravity sensing devices 18 and 20, angled to each other, and with a plane containing the superposition of the respective axes of sensitivity of gravity sensing devices 18 and 20 oriented at an angle to the axial portion of the patient's body. Right angles between the axes of sensitivity of gravity sensing devices 18 and 20, and between the plane containing the superposition of those axes and the axis of the axial portion of the patient's body normally give the greatest theoretical sensitivity. However, other angles will work as long as they are sufficient to let the position sensor 16 (or a device receiving information from position sensor 16) determine which of four (or three if desired) positions sensor 16 is in, and to correlate those positions to the positions of the patient's body.

In a preferred embodiment, the housing 12 contains the vibration sensor 14 and the position sensor 16 so that both sensors may be coupled to the patient simultaneously in a single step and are substantially adjacent to one another since both are contained within housing 12. However, while this is simpler, it is not essential to the present invention, and vibration sensor 14 and position sensor 16 may be applied to the patient in substantially adjacent locations in two different steps if desired.

In a preferred embodiment, an approximately cylindrical housing is used, but again the shape of the housing is often not critical. Preferably, the housing should not cause discomfort nor interfere with sleep, should not be difficult to handle, and should be coupled to the patient so as to keep the sensors reliably in place over a sleep period.

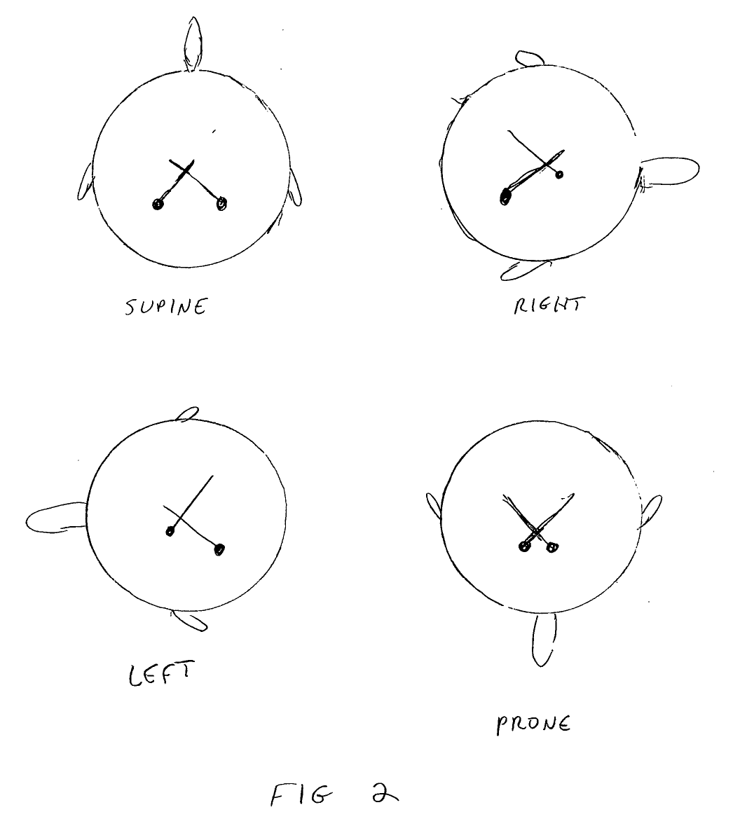

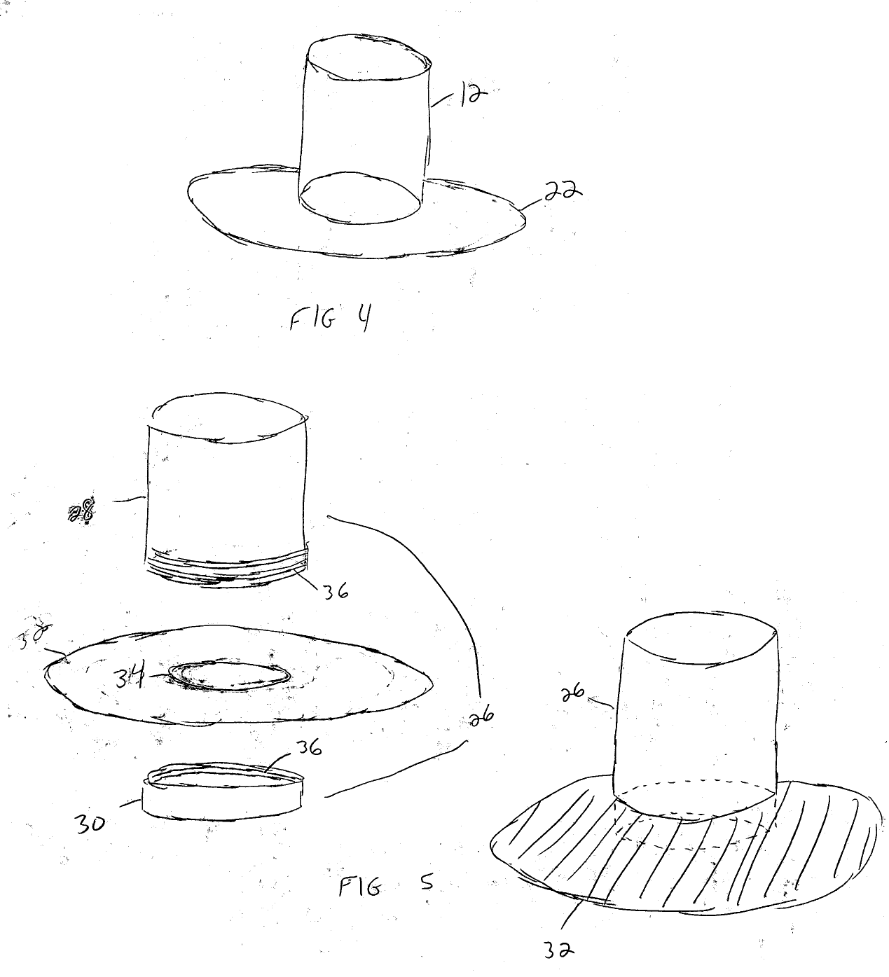

In one preferred embodiment, coupling the housing to the patient may be easily accomplished by using adhesive, and in particular the adhesive may be removably coupled to the housing, so that the housing may be conveniently reused with new adhesive, rather than directly attaching adhesive to the housing or the sensors. One embodiment of such a removable adhesive is an adhesive patch 22 as shown in FIG. 3. Adhesive patch 22 is shown as a circular patch with a circular layer of adhesive 24 in the center of the top of the patch. The layer of adhesive is the size of the outside edge of housing 12. As shown in FIG. 3B, the entire bottom of adhesive patch 22 is covered with adhesive. Not shown in FIG. 3B is a protective peel-away covering to protect the adhesive surface underneath. This peel-away component may have a tab or other means to facilitate the peeling action. As shown in FIG. 4, adhesive patch 22 is attached to housing 12 by means of the adhesive on the top of patch 22. The bottom side of adhesive patch 22 is then applied to the patient at a preferred location as described above, thus holding housing 12 in the desired position. The use of the patch 22 makes it easier to replace the adhesive after each use and reuse the device on another patient. If desired, a hole may be located in the center of adhesive patch 22 that is slightly smaller than the outside edge of housing 12, so that the material of patch 22 does not interfere with the sensitivity of vibration sensor 14. In this case, the top of adhesive patch 22 will have adhesive only on the portion of patch 22 surrounding the central hole that will come in contact with housing 12. Yet another possibility is a patch that wraps up the side of housing 12, with adhesive on the portion of the top side of the patch that contacts the side and bottom of housing 12.

Alternative methods of attachment are also possible. For example, as shown in FIG. 5, one could use a housing 26, which has two circular components that screw together (28 and 30), and an adhesive patch 32 that has adhesive only on the bottom. Adhesive patch 32 again has a circular hole 34 in its center that is slightly smaller than an outer diameter of housing 26, and is attached to housing 26 by screwing the two components 28 and 30 together with the center of adhesive patch 32 in between them. The portion of adhesive patch 32 that is smaller than an outer diameter of housing 26 is thus held between the housing components 28 and 30, and the adhesive on the bottom of patch 32 thus again allows the housing to be attached to the patient.

In addition to these methods, an adhesive patch could be attached to an object that couples to the housing, e.g. a flat panel that screws onto a portion of the housing. As additional examples, a housing, or the separate sensors 14 and 16 could be attached directly to the patient by implantation, suction, suturing, or needling. The sensors could be also be coupled to the patient by their attachment to a collar that goes substantially around the patient's neck, the ends of the collar being held together by a clasp or a hook and loop material such as Velcro, or the collar could be made of a bendable plastic which is flexible enough to be opened to place it on the neck but stiff enough to remain on the patient during sleep. A collar coupling may be particularly useful in veterinary applications to forestall dislodgement by an animal patient.

As described above, the position sensor 16 can be completely contained within housing 12. It is also possible that some portion of position sensor 16 can be located outside the housing, for example in a cable attached to the housing. In a preferred embodiment, there is an indicator on the housing that shows the patient the direction in which the housing 12 is to be oriented when he or she attaches it to himself or herself. Also in a preferred embodiment, this indicator is a protrusion from the housing that is to be pointed down the patient's body from the pre-tracheal location when the housing 12 is attached. The indicator may also be one or more labels, icons (e.g. feet icons to show the inferior direction, and head or eye icons to show the superior direction), arrows or other marks on the housing, but a protrusion is preferred since the patient may not be able to see the pre-tracheal location well unless he or she is looking in a mirror when the housing is attached. The indicator may fulfill a plurality of functions, e.g. a ridge on the housing could serve as an indicator and as a means to facilitate gripping of the housing. The indicator could alternatively or additionally be a color or texture or shape property of a portion of the housing. The shape, coloring, texture, or other property(s) of an object coupled to the housing, e.g. the adhesive, could be used to indicate proper placement and/or orientation of the housing 12.

The information from vibration sensor 14 and information from position sensor 16 are preferably obtained concurrently, over a period of sleep or diminished consciousness. Here, “concurrently” is defined to mean either simultaneously or in an alternating fashion. If the information from each sensor is obtained in an alternating fashion, the periods during which vibration information is not obtained (i.e., periods when orientation information is obtained) should be relatively short, preferably under 10 seconds. This is because most breaths last less than 10 seconds and longer periods of not obtaining tracheal vibration information are likely to carry a risk of missing tracheal vibration information useful in characterizing the patient's respirations. Further, to obtain sufficient information to do a proper evaluation, in a preferred embodiment the total period over which information is obtained should be of a substantial duration such as at least approximately 6 hours.

While the information from vibration sensor 14 may be recorded in analog form, such as on recording tape, in a preferred embodiment the information is converted into digital data by an analog-to digital (A/D) converter. In keeping with the desired frequency range of vibration information in one embodiment of the present invention of approximately 400 to 1000 Hz, i.e. 1 KHz, the sampling rate must be at least double the highest frequency to be sampled in order to obtain a reliable sample, and thus the sampling rate must be at least 2 KHz in such a preferred embodiment.

In a preferred embodiment, the position information consists of 4 voltage levels, representing the 4 positions being recorded, although an embodiment with more position discrimination could be constructed and would still be within the present invention. These voltage levels are sampled twice per second, and the resulting sample is digitized by an A/D converter.

The digital data representing the collected information is then stored for later analysis. In a preferred embodiment, it is stored by a recording device in a non-volatile memory so that once it is stored no further power is needed to preserve the integrity of the data. Using the 2 KHz sampling rate, data resolution of 12 bits to allow for dynamic range, and a period of 6 hours, it is estimated that a memory of 64 megabytes (MB) should be sufficient to store the digital data. (Note that a data resolution of 12 bits is generally equivalent to a dynamic range of approximately 72 decibels.) It is, however, possible that a lower sampling rate or data resolution, application of a compression algorithm to the data, or a shorter time, could be used, thus reducing the amount of memory required.

Flash memory is a preferred non-volatile memory, due to its low cost and size, but other media could also be used, including, but not limited to, magnetic RAM (random access memory), small hard or floppy disk drives, or audio or video tape.

In a preferred embodiment, the recording device and the non-volatile memory are contained in a small box that is attached to the patient's body, e.g. by a wrist strap. In addition to components already mentioned, the box may contain additional components such as a programmable processing unit (“CPU”), hardware filters, communication hardware and software (e.g. a “USB” communication capability), and the like. The box is preferably small and light enough so that, once attached to the patient, it will not interfere with sleep. A small, self-contained power source may also be contained within the box to power the recording device. A cable, containing one or more wires, brings the information from housing 12, and thus from vibration sensor 14 and position sensor 16, to the recording device. Alternatively, the information may be transmitted wirelessly from housing 12 to the recording device. An A/D converter that converts the information from vibration sensor 14 may be located in or adjacent to the housing, or it may be contained within the box.

As yet another alternative, the recording means may be in a base station near the patient, and the information again transmitted wirelessly for recording. In this case, the size of the base station is not important, and no self-contained power source is needed for the base station, as the base station may be plugged into a normal AC outlet. Again, an A/D converter may be located in or near the housing 12 on the patient, in the base station with the recording means, or even within a cable. It is seen, therefore, that there is an embodiment of the invention in which the patient is not encumbered by any cables at all: the invention could be entirely self-contained in one or more small housings, coupled to the patient, for example, by a simple peel-and-stick adhesive component.

In some embodiments a mobile telephone could serve as a base station to transmit to a remote recording or analysis means. In such an embodiment, an adapter may plug into the phone to deliver data derived from the sensors.

Another possible addition is an audio playback means, so that the vibration information may be recreated to allow a person to listen to it. In a preferred embodiment, the playback means should have a frequency range at least approximately equal to the frequency sensitivity range of vibration sensor 14 so that effectively all of the captured vibration information per unit time is reproduced. In a preferred embodiment, the listener should be able to hear at least substantially what he or she would have heard had he or she been listening to the patient's tracheal sounds through a stethoscope located at the same position on the patient's body as the vibration sensor. In some cases, processing the captured vibration data before playback may, mathematically, reduce the amount of information in the playback, but may render it more perspicuous for the listener. For example, it is possible to filter vibration information to emphasize or de-emphasize certain classes of sounds, e.g. filtering out low-frequency heart sounds might allow a listener to better appreciate respiratory sounds during playback.

Various transforms may be applied to data to generate various representations of the collected information, including, but not restricted to filtering, scaling, compression, Fourier transforms, Hartley transforms, wavelet transforms, etc.

Other types of sensors may be added to the basic invention to increase accuracy or increase information yield. For example, one might add a component enabling the sensing of within-body gasses (e.g. oxygen as measured by oximetry, carbon dioxide measured transcutaneously), a component for measuring the patient's temperature (e.g. skin temperature), a component for measuring body motion with more sensitivity than just position changes (e.g. actigraphy), a component for measuring light in the patient's sleeping environment, a component for measuring respiratory effort (Meslier. Sleep. 2002; 25:753-7.), and so on. Such information, if properly analyzed, could improve the ability of the device to diagnose and/or characterize OSA, other types of SDB, and other types of sleep disorders. However, the peri-tracheal location used in the present invention may not be appropriate for some sensors, or they may not easily fit within a housing appropriate for that location, thus requiring that such other sensors be located elsewhere, and that they have separate leads or other means for sending their data to the recording device.

For example, combining a version of the invention having wireless data transmission capability, sited peri-tracheally and containing a power source, with an oximeter, sited on a finger or elsewhere, also having wireless data transmission and containing a power source, could provide a substantial amount of information about a patient's cardiorespiratory status without encumbering the patient with cables of any kind, again with a base station for receiving information.

It may also be advisable for the sensor to provide an identification code so the data recorder knows what kind of data to expect, and can record it in the appropriate format. Providing such codes may find utility when more than one type of sensor is in use. For example, sensor type “A” may provide data related to tracheal vibration and body-position, sensor type “B” may provide the same as “A” but be sized for children and require a different analysis algorithm, sensor “C” may provide information related to tracheal vibration plus body position plus temperature, and so on. Identification codes could contain information about manufacturer lot number, individual serial numbers, etc. The identification code could be conveyed via various means, e.g. a cable or wirelessly, and could even be implemented using radiofrequency identification chips (See, e.g. Booth-Thomas. Time. 2003 Sep. 22).

As noted above, the present invention has several advantages over the existing art. There is only one sensor connection to the patient's body to be concerned about, so that the patient is more likely to be able to apply the sensor housing to himself or herself correctly and without assistance. With only one sensor housing, the device should be less likely to interfere with sleep, and positioning the sensor housing in a peri-tracheal location is likely to be less disturbing to sleep in some people than is placement in other locations, such as on the head or face. If the proper adhesive (or other attachment means) is used, the sensor housing is unlikely to be dislodged during sleep, so that an attendant is not needed. In general, minimizing the size of the housing may be expected increase both comfort and resistance to dislodgement. We have found, for example, that a vertical dimension of 2 centimeters for the sensor housing does not substantially interfere with sleep in adults or children down to age 6. Thus, we expect that a reduced-sensor device employing the combination position/vibration sensor of the current invention as its only sensor attachment, will find wide applicability in the evaluation of OSA and SDB.

Although the foregoing specification has referred primarily to the use of the invention with regard to a sleeping patient, the invention may find use in patients having other types of diminished consciousness, including, but not restricted to states of coma or stupor, or after the administration of a sedative medication or a general anesthetic, etc.

In the foregoing specification, the invention has been described with reference to specific embodiments thereof. It will, however, be evident to one of ordinary skill in the art that various modifications and changes can be made thereto without departing from the broader spirit and scope of the invention as set forth in the appended claims. For example, different shaped housings may be used, the vibration sensor may detect various frequency ranges, and position sensors of different shapes or types or having a different number of degrees of orientation may be used. The specification and drawings are, accordingly, to be regarded in an illustrative rather than a restrictive sense. Therefore, the scope of the invention should be limited only by the appended claims.

1. A system for monitoring a patient, comprising:
vibration sensing means for collecting tracheal vibration information from the patient; and
position sensing means that changes state depending upon its orientation with respect to the earth's gravity, at least a portion of which is substantially adjacent to a portion of the vibration sensing means.

2. The system of claim 1, wherein the vibration sensing means comprises a microphone.

3. The system of claim 1, wherein the position sensing means comprises an accelerometer.

4. The system of claim 1, wherein the position sensing means comprises a gravity sensing switch having at least one axis of orientation with respect to gravity such that the switch occupies different states depending upon which end of the axis is closer to the source of gravity.

5. The system of claim 4, wherein the gravity sensing switch further comprises a tilt switch having:
a body containing a cavity;
a plurality of contact point pairs within the cavity;
an electrically conductive material that is able to move within the cavity, such that as the orientation of the body with respect to gravity changes different pairs of contact points are connected, thus providing a signal indicative of the tilt switch's orientation with respect to gravity.

6. The system of claim 4, wherein the position sensing means comprises:
a first gravity sensing switch having a first axis of orientation with respect to gravity; and
a second gravity sensing switch having a second axis of orientation with respect to gravity which can be superpositioned at an angle to the first axis.

7. The system of claim 6, further comprising a means for coupling the system to at least a portion of the patient's body, such that the position sensing means provides information indicative of changes in orientation of the portion of the patient's body to which the system is coupled.

8. The system of claim 7, wherein a plane containing the superposition of the two axes is at an angle to the portion of the patient's body such that the position sensing means provides information indicative of which of two or more positions the portion of the patient's body is in with respect to the earth's gravity.

9. The system of claim 8, wherein the portion of the patient's body to which the housing is coupled, and to which the plane is at an angle, is an axial portion.

10. The system of claim 9, wherein the angle between the two superpositioned axes is substantially a right angle.

11. The system of claim 10, wherein the angle between the plane and the axial portion of the patient's body is substantially a right angle.

12. The system of claim 1, further comprising means for simultaneously coupling at least a portion of the vibration sensing means and a portion of the position sensing means to a portion of the patient's body, such that the position sensing means tracks changes in orientation of the portion of the patient's body to which the system is coupled.

13. The system of claim 12, wherein the means for simultaneously coupling further comprises a housing containing at least a portion of the vibration sensing means and a portion of the position sensing means.

14. The system of claim 13, wherein the means for simultaneously coupling further comprises an adhesive material coupled to a portion of the housing.

15. The system of claim 9, wherein the angle between the superpositioned axes of the two tilt switches, and the angle between the plane and the axial portion of the patient's body, are such that the tilt switches indicate which of two or more positions the axial portion of the patient's body is in, one of which positions is substantially supine and one of which positions is not substantially supine.

16. The system of claim 9, wherein the angle between the superpositioned axes of the two tilt switches, and the angle between the plane and axial portion of the patient's body, are such that the sensor indicates which of four or more positions the axial portions of the patient's body is in, one of which positions is substantially supine, one of which positions is substantially prone, one of which positions is substantially left lateral decubitus, and one of which positions is substantially right lateral decubitus.

17. The system of claim 4, further comprising means for coupling the system to an axial portion of the patient's body, with the axis of the tilt switch at an angle to the axial portion such that the tilt switch provides information indicative of which of two or more positions the axial portion of the patient's body is in, one of which positions is substantially supine and one of which positions is not substantially supine.

18. The system of claim 1, further comprising a recording means for recording data representing the tracheal vibration information and data representing the state of the position sensing means over time.

19. The system of claim 13, further comprising a recording means for recording data representing the tracheal vibration information and data indicative of the orientation of the portion of the patient's body to which the system is coupled over time.

20. The system of claim 19, further comprising a sampling means capable of sampling the tracheal vibration information at a rate of at least 2 kilohertz.

21. The system of claim 19, wherein the recording means further comprises:
a memory;
a power source,
conversion means for receiving the tracheal vibration information and the information indicative of the orientation of the patient's body and converting them into digital data; and
means for writing the digital data into the memory.

22. The system of claim 21, wherein the memory further comprises a memory capable of storing 32 megabytes of data.

23. The system of claim 21, wherein an input of the conversion means is connected to an output of at least one of the sensing means by a wireless transmitter and receiver, where the transmitter is connected to an output of the sensing means and the receiver is connected to an input of the conversion means.

24. The system of claim 21, wherein an output of the conversion means is connected to an input of the memory by a wireless transmitter and receiver, where the transmitter is connected to the output of the conversion means and the receiver is connected to an input of the memory.

25. The system of claim 21, further comprising a playback means capable of substantially recreating the collected tracheal vibration information from the recording means.

26. The system of claim 25, wherein the vibration sensing means further comprises a microphone having a frequency response of at least approximately 400 to 1000 hertz, and the playback means further comprises a sound output device capable of reproducing sound in a range of at least approximately 400 to 1000 hertz, such that upon playback of the data representing the collected tracheal vibration information a listener hears at least substantially the same sound that the listener would have heard through a listening device having a frequency response of at least approximately 400 to 1000 hertz in the same position as the vibration sensing means at the time the tracheal vibration information was collected.

27. The system of claim 25, wherein the vibration sensing means further comprises a microphone having a frequency response, and the playback means further comprises a sound output device capable of reproducing sound in a range of at least approximately the same frequency response at the microphone, such that upon playback of the data representing the collected tracheal vibration information a listener hears at least substantially the same sound that the listener would have heard through a listening device having approximately the same frequency response as the microphone in the same position as the microphone at the time the tracheal vibration information was collected.

28. The system of claim 25, wherein the vibration sensing means further comprises a microphone having a frequency response containing a portion of the range of 400 to 1000 hertz, and the playback means further comprises a sound output device capable of reproducing sound in the same portion of the range of 400 to 1000 hertz, such that upon playback of the data representing the collected tracheal information a listener hears at least substantially the same sound that the listener would have heard at the time the tracheal vibration information was collected through a listening device having approximately the same frequency response as the microphone in a peri-tracheal position on the patient.

29. The system of claim 19, further comprising a computing device for reading and performing calculations on the recorded data.

30. The system of claim 13, further comprising an indicator means on the housing for showing the orientation the housing is to have when coupled to the patient's body.

31. A method for monitoring a patient, comprising:
collecting tracheal vibration information from the patient at a location on the patient's body; and
obtaining information indicative of the orientation of a portion of the patient's body with respect to gravity substantially adjacent to the location at which the tracheal vibration information is collected.

32. A method for monitoring a patient, comprising:
coupling to the patient a vibration sensing means for collecting tracheal vibration information from a patient; and
coupling to at least a portion of the patient's body, substantially adjacent to the vibration sensing means, a position sensing means that changes state depending upon its orientation with respect to gravity, such that the position sensing means provides information that is indicative of the orientation with respect to gravity of the portion of the patient's body to which it is coupled.

33. The method of claim 32, further comprising the step of recording data representing tracheal vibration information and information indicative of the orientation of the portion of the patient's body that are obtained over time.

34. The method of claim 33, wherein the step of recording data further comprises recording data from both the vibration sensing means and the position sensing means that are obtained concurrently.

35. The method of claim 33, wherein the step of recording data further comprises recording data during a period of time associated with diminished consciousness of the patient.

36. The method of claim 32, wherein the step of coupling a vibration sensing means further comprises coupling a microphone to the patient.

37. The method of claim 32, wherein the step of coupling to the patient a vibration sensing means further comprises coupling said means near a tracheal segment of the patient.

38. The method of claim 32, wherein the step of coupling a position sensing means to the patient further comprises coupling an accelerometer.

39. The method of claim 32, wherein the step of coupling a position sensing means to the patient further comprises coupling to a portion of the patient's body a gravity sensing device having at least one axis of orientation with respect to gravity such that the gravity sensing device occupies different states depending upon which end of the axis is closer to the source of gravity.

40. The method of claim 39, wherein the gravity sensing device is coupled to an axial portion of the patient's body with the axis of orientation of the gravity sensing device at an angle to the axial portion such that the gravity sensing device provides information indicative of which of two or more positions the axial portion of the patient's body is in, one of which positions is substantially supine and one of which positions is not substantially supine.

41. The method of claim 39, wherein step of coupling a position sensing means to the patient further comprises coupling:
a first gravity sensing device having a first axis of orientation with respect to gravity; and
a second gravity sensing device having a second axis of orientation with respect to gravity which can be superpositioned at an angle to the first axis.

42. The method of claim 41, wherein the step of coupling a position sensing means to the patient further comprises coupling the gravity sensing devices to an axial portion of the patient's body with a plane containing the superposition of the two axes at an angle to an axial portion of the patient's body such that the states of the gravity sensing devices provide information indicative of which of two or more positions the axial portion of the patient's body is in.

43. The method of claim 41, wherein the step of coupling a position sensing means to the patient further comprises coupling the gravity sensing devices to an axial portion of the patient's body with the angle between the superposition of the two axes, and the angle between the plane containing the gravity sensing devices and the axial portion of the patient's body, being such that the states of the gravity sensing devices provide information indicative of which of two or more positions the axial portion of the patient's body is in, one of which positions is substantially supine and one of which positions is not substantially supine.

44. The method of claim 41, wherein the step of coupling a position sensing means to the patient further comprises coupling the gravity sensing devices to an axial portion of the patient's body with the angle between the axes of the two gravity sensing devices, and the angle between the plane containing the gravity sensing devices and long axis of the patient's body, being such that the states of the gravity sensing devices provide information indicative of which of three or more positions the axial portion of the patient's body is in, one of which positions is substantially supine, one of which positions is substantially prone, and one of which positions is one or more of the substantially lateral decubitus positions of the patient.

45. The method of claim 41, wherein the step of coupling a position sensing means to the patient further comprises coupling the gravity sensing devices to an axial portion of the patient's body with the angle between the superpositioned axes of the two gravity sensing devices, and the angle between the plane and axial portion of the patient's body, being such that the states of the gravity sensing devices provide information indicative of which of four or more positions the axial portion of the patient's body is in, one of which positions is substantially supine, one of which positions is substantially prone, one of which positions is left lateral decubitus, and one of which positions is right lateral decubitus.

46. The method of claim 41, wherein the step of coupling a position sensing means to the patient further comprises coupling to the patient a housing containing the vibration sensing means and the first and second gravity sensing devices.

47. The method of claim 41, wherein the first gravity sensing device further comprises a tilt switch having:
a body containing a cavity;
a plurality of contact point pairs within the cavity;
an electrically conductive material that is able to move within the cavity, such that as the orientation of the body with respect to gravity changes different pairs of contact points are connected, thus providing a signal indicative of the switch's orientation with respect to gravity.

48. The method of claim 41, wherein the first gravity sensing device is an accelerometer.

49. The method of claim 34, wherein the step of recording data representing the tracheal vibration and orientation information further comprises the steps of:
providing a memory;
converting the tracheal vibration information and information indicative of the orientation of the portion of the patient's body into digital data; and
writing the digital data into the memory.

50. The method of claim 49, wherein the step of providing a memory further comprises providing a non-volatile memory, and further comprises the step of:
coupling the non-volatile memory to the patient such that the patient may be in a state of diminished consciousness without being disturbed during the period of diminished consciousness.

51. The method of claim 49, wherein the step of recording data further comprises the step of:
wirelessly transmitting the tracheal vibration information and information indicative of the orientation of the portion of the patient's body from the sensing means to a recording device containing a memory before the step of converting the data into digital data.

52. The method of claim 49, wherein the step of recording data further comprises the step of:
wirelessly transmitting the digital data to a recording device containing a memory between the steps of converting the information into digital data and the step of writing the digital data into the memory.
